(19) United States
(12) Patent Application Publication
(10) Pub. No.: US 2021/0292403 A1
(43) Pub. Date: Sep. 23, 2021

(54) THERAPIES FOR TREATING PROCTITIS WITH POLYCLONAL ANTI-TNFALPHA ANTIBODIES

(71) Applicant: MICROPHARM LIMITED, Newcastle Emlyn (GB)

(72) Inventors: John Landon, Newcastle Emlyn (GB); Ruth Elizabeth Coxon, Newcastle Emlyn (GB)

(21) Appl. No.: 17/263,789

(22) PCT Filed: Jul. 29, 2019
(86) PCT No.: PCT/GB2019/052121

§ 371 Date: Jan. 27, 2021

(30) Foreign Application Priority Data
Jul. 27, 2018 (GB) 1812262.2

(51) Int. Cl. C07K 16/24; A61K 47/42; A61K 47/44; A61K 47/34; A61K 9/02; A61K 9/00; A61P 1/00

(52) U.S. Cl. CPC C07K 16/241; A61K 47/42; A61K 47/44; A61K 47/34; A61K 2039/505; A61K 9/0031; A61P 1/00; C07K 2317/20; C07K 2317/76; A61K 9/02

(57) ABSTRACT

The present invention is directed to an antibody composition for use in treating proctitis, wherein the antibody composition comprises intact blood-derived polyclonal antibodies that bind to a human tumour necrosis factor α (TNFα). The invention also relates to associated uses, methods, solid compositions, and liquid compositions using/comprising the same.

The present invention relates to an antibody therapeutic suitable for use in treating proctitis.

Tumour necrosis factor α (TNFα) is a principal cytokine mediating systemic inflammation, and is implicated in a number of diseases and disorders including proctitis.

Current antibody therapeutics rely on systemic administration of monoclonal antibodies directed against TNFα. Such monoclonal antibodies are typically chimeric or humanised with a view to avoiding induction of a humoral immune response in the patient. The use of polyclonal antibodies of animal origin has been avoided due to the risk of triggering such a response. Three monoclonal antibodies currently in widespread use are the chimeric murine Infliximab, and fully humanised Adalimumab and Etanercept. Infliximab is infused intravenously while Adalimumab and Etanercept are infused subcutaneously.

Systemic use of antibodies has been associated with infusion reactions and such treatment is inconvenient for out-patients since intravenous infusions usually require a short stay in hospital. Additionally, the efficiency with which a systemically administered McAb (Molecular Weight ˜150,000 Da) will pass from the blood to the tissue fluid and then reach the inflamed lining of the rectum/anus is also questionable. Moreover, since TNFα is a cytokine that plays an important pro-inflammatory role systemically in protecting patients from infection, long-term systemic administration of anti-TNFα antibodies is associated with an increased incidence of serious side-effects including reactivation of tuberculosis, opportunistic infections, demyelinating diseases and a long term risk of lymphoma.

Proctitis is an inflammation of the anus and the lining of the rectum. Symptoms of proctitis can be diverse including a continual urge to defecate, diarrhoea, ineffectual straining to empty the bowels, spasms and cramping during defecation, pain and irritation in the rectum and anal region (including pain during defecation), bleeding, and discharge. The causes of proctitis can be diverse. For example, in some cases proctitis can be idiopathic, while in other cases it may arise as a result of undergoing chemotherapy or radiotherapy (radiation proctitis), a sexually transmitted infection (e.g. herpes proctitis) or as a result of coeliac disease.

Current therapies for proctitis include oral anti-inflammatories taken by mouth, suppository or enema, such as mesalazine (Asacol) or corticosteroids (e.g. prednisone or budesonide (Entocort® EC)). In serious cases proctitis may be treated by surgery.

The conventional proctitis therapies do not target the underlying pathology. Thus, there is a need for a therapeutic targeting the mechanism of proctitis as opposed to only providing symptomatic relief.

The present invention solves at least one of the above-mentioned problems.

The present inventors have surprisingly found that intact blood-derived antibodies of the invention that bind TNFα constitute an improved therapeutic for proctitis.

Polyclonal TNFα antibodies derived from blood as per the present invention surprisingly exhibit improved efficacy and improved specific titres when compared to conventionally manufactured antibodies (e.g. milk-derived antibodies). An advantage of this is that the antibodies of the present invention require very little further processing (e.g. affinity purification) prior to being able to be used for therapeutic purposes, thereby reducing costs associated with production of said antibodies (e.g. compared to monoclonal antibodies or even polyclonal antibodies from alternative sources). Thus, in a preferable embodiment, a polyclonal antibody of the invention or a composition comprising polyclonal antibodies of the invention has not been subjected to extensive purification, more preferably a polyclonal antibody of the invention or a composition comprising polyclonal antibodies of the invention has not been subjected to affinity purification (using TNFα, such as human TNFα).

Additionally, or alternatively polyclonal antibodies of the invention when administered rectally to a subject elicit no, or greatly-reduced, side-effects (e.g. humoral immune response side-effects) when compared to conventional antibodies (e.g. monoclonal antibodies) given systemically. Hence, the composition of the invention is suitable for prolonged therapeutic use, unlike systemically administered antibody compositions.

As a further advantage, manufacture of said polyclonal antibodies is much less expensive than conventional monoclonal antibodies. Hence, the present invention provides a scalable and/or cost-efficient therapeutic.

In one aspect the invention provides an antibody composition for use in treating proctitis, wherein the antibody composition comprises intact blood-derived polyclonal antibodies that bind to a human tumour necrosis factor α (TNFα).

In another aspect there is provided use of an antibody in the manufacture of a medicament for treating proctitis, wherein the antibody is an intact blood-derived polyclonal antibody that binds to a human tumour necrosis factor α (TNFα).

In a related aspect the invention provides a method of treating proctitis, the method comprising administering an antibody composition to a subject, wherein the antibody composition comprises intact blood-derived polyclonal antibodies that bind to a human tumour necrosis factor α (TNFα).

Advantageously, blood-derived polyclonal antibodies to TNFα can be obtained multiple times from the blood of the same host without killing said host. This is in contrast to conventional methods using sources such as bovine colostrum, which only yields antibodies for a limited time (e.g. once).

The blood-derived antibodies may be obtainable from a non-human mammal.

The term “obtainable” as used herein also encompasses the term “obtained”. In one embodiment the term “obtainable” means obtained.

Preferably the antibodies are ovine or equine polyclonal antibodies.

More preferably, the antibodies are ovine polyclonal antibodies (e.g. obtainable from an ovine).

Thus, in one embodiment there is provided an antibody composition comprising intact ovine polyclonal antibodies that bind to a human tumour necrosis factor α (TNFα).

An antibody of the present invention binds to and neutralises human tumour necrosis factor α (TNFα). The antibody binds to human TNFα with a higher binding affinity than for a non-human TNFα or an alternative antigen. In one embodiment an antibody of the invention binds to human TNFα with an affinity (measured by the dissociation constant: K_{d}) of at least 10⁻⁴M or at least 10⁻⁵M. In one embodiment an antibody of the invention may bind to human TNFα with an affinity (K_{d}) of at least 10⁻⁶ M or 10⁻⁷ M. Suitably an antibody of the invention may bind to human TNFα with an affinity (K_{d}) of at least 10⁻⁸M or 10⁻⁹M.

Alternatively or additionally, antibody binding affinity may be measured by way of the association constant (Kₐ). In one embodiment an antibody of the invention binds to human TNFα with an affinity (measured by the association constant: Kₐ) of at least 10⁶ M. Suitably an antibody of the invention binds to human TNFα with an affinity (measured by the association constant: Kₐ) of at least at least 10⁷M (e.g. at least 10⁸ M).

Antibody binding can be tested using the assay described in Example 3. Neutralisation can be tested using the assay described in Example 4. In more detail, an antibody composition can be assayed to test neutralisation of the cytotoxic effect of TNFα on a L929 mouse fibrosarcoma cell line. L929 cells may be commercially available from LGC Standards, UK, cat. no. ATCC®CCL-1™.

Surprisingly, blood-derived antibodies obtainable from a non-human mammal (preferably an ovine and/or an equine non-human mammal) immunised with human TNFα (or a purified fraction thereof) contains a much higher concentration of antibodies specific for human TNFα, when compared to alternative sources (e.g. avian [for example from egg yolk] or bovine sources, such as milk). In some cases the concentration of specific antibodies in said ovine blood (e.g. serum) or purified fraction thereof is 100 times greater.

In one embodiment at least 5% (suitably at least 10%) of the total antibodies comprised in a blood sample obtainable from a host (e.g. non-human mammal) immunised with human TNFα (or a purified fraction thereof) binds to human TNFα. In another embodiment at least 15% of the total antibodies comprised in a blood sample obtainable from a host (e.g. non-human mammal) immunised with human TNFα (or a purified fraction thereof) binds to human TNFα. In another embodiment at least 20% of the total antibodies comprised in a blood sample obtainable from a host (e.g. non-human mammal) immunised with human TNFα (or a purified fraction thereof) binds to human TNFα.

In one embodiment at least 5% (suitably at least 10%) of the total antibodies comprised in a blood sample obtainable from an ovine host immunised with human TNFα (or a purified fraction thereof) binds to human TNFα. In another embodiment at least 15% of the total antibodies comprised in a blood sample obtainable from an ovine host immunised with human TNFα (or a purified fraction thereof) binds to human TNFα. In another embodiment at least 20% of the total antibodies comprised in a blood sample obtainable from an ovine host immunised with human TNFα (or a purified fraction thereof) binds to human TNFα.

In one embodiment at least 5% (suitably at least 10%) of the total antibodies comprised in a blood sample obtainable from an equine host immunised with human TNFα (or a purified fraction thereof) binds to human TNFα. In another embodiment at least 15% of the total antibodies comprised in a blood sample obtainable from an equine host immunised with human TNFα (or a purified fraction thereof) binds to human TNFα. In another embodiment at least 20% of the total antibodies comprised in a blood sample obtainable from an equine host immunised with human TNFα (or a purified fraction thereof) binds to human TNFα.

The antibodies referred to above also preferably neutralise said human TNFα.

In one embodiment at least 5% (suitably at least 10%) of the total antibodies comprised in a blood sample obtainable from an ovine host immunised with human TNFα (or a purified fraction thereof) binds to and neutralises human TNFα. In another embodiment at least 15% of the total antibodies comprised in a blood sample obtainable from an ovine host immunised with human TNFα (or a purified fraction thereof) binds to and neutralises human TNFα. In another embodiment at least 20% of the total antibodies comprised in a blood sample obtainable from an ovine host immunised with human TNFα (or a purified fraction thereof) binds to and neutralises human TNFα.

In one embodiment at least 5% (suitably at least 10%) of the total antibodies comprised in a blood sample obtainable from an equine host immunised with human TNFα (or a purified fraction thereof) binds to and neutralises human TNFα. In another embodiment at least 15% of the total antibodies comprised in a blood sample obtainable from an equine host immunised with human TNFα (or a purified fraction thereof) binds to and neutralises human TNFα. In another embodiment at least 20% of the total antibodies comprised in a blood sample obtainable from an equine host immunised with human TNFα (or a purified fraction thereof) binds to and neutralises human TNFα.

In one embodiment an antibody composition comprises intact polyclonal antibodies at a concentration of 10-1000 mg. Preferably, an antibody composition comprises intact polyclonal antibodies at a concentration of 10-500 mg, such as 10-250 mg. More preferably, an antibody composition comprises intact polyclonal antibodies at a concentration of about 50-100 mg. The concentrations referred to may be the concentrations of total intact polyclonal antibodies, suitably total intact polyclonal IgG (i.e. including antibodies that do, as well as do not, bind to TNFα). In one embodiment the foregoing embodiments refer to intact polyclonal antibodies that bind to (and preferably neutralise) TNFα.

Alternatively, an antibody composition may comprise intact polyclonal antibodies at a concentration of 1-100 g/L, such as 2-50 g/L. Preferably antibodies may be present at a concentration of about 20-75 g/L. More preferably, an antibody composition comprises intact polyclonal antibodies at a concentration of about 50 g/L. The concentrations referred to may be the concentrations of total intact polyclonal antibodies, suitably total intact polyclonal IgG (i.e. including antibodies that do, as well as do not, bind to TNFα). In one embodiment the foregoing embodiments refer to intact polyclonal antibodies that bind to (and preferably neutralise) TNFα.

An “antibody” is a protein including at least one or two, heavy (H) chain variable regions (abbreviated herein as VHC), and at least one or two light (L) chain variable regions (abbreviated herein as VLC). The VHC and VLC regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. Sequences of Proteins of Immunological Interest, Fifth Edition, U.S. Department of Health and Human Services, NIH Publication No. 91-3242, 1991, and Chothia, C. et al, J. Mol. Biol. 196:901-917, 1987, which are incorporated herein by reference). Preferably, each VHC and VLC is composed of three CDRs and four FRs, arranged from amino-terminus to carboxy-terminus in the following order: FR1, CDR1, FR2, CDR2, FR3, CDR3, FR4.

The VHC or VLC chain of the antibody can further include all or part of a heavy or light chain constant region. In one embodiment, the antibody is a tetramer of two heavy immunoglobulin chains and two light immunoglobulin chains, wherein the heavy and light immunoglobulin chains are inter-connected by, e.g., disulphide bonds. The heavy chain constant region includes three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system. The term “antibody” includes intact immunoglobulins of types IgA, IgG, IgE, IgD, IgM (as well as subtypes thereof), wherein the light chains of the immunoglobulin may be of types kappa or lambda. Preferably an antibody of the invention is an intact IgG.

The term “intact antibody” is used herein to distinguish an antibody of the invention from an antibody fragment (e.g. an antibody Fab, F(ab)₂ or Fc). An “intact antibody” therefore comprises (or consists of) each of the antibody regions/domains present in a full-length antibody obtainable from a non-human mammal described herein (e.g. ovine or equine, preferably ovine). A monomer of an “intact antibody” comprises (or consists of) two heavy chains, and two light chains. The heavy chains each comprise (or consist of) a VH domain, a CH1 domain, a CH2 domain, and a CH3 domain. The light chains each comprise (or consist of) a CL domain and a VL domain.

Thus, an antibody composition of the present invention comprises no or substantially no antibody fragments (e.g. Fab, F(ab)₂ or Fc fragments). The term “substantially no” as used in this context means that less than 5%, 4%, 3%, 2%, 1%, 0.5%, 0.1%, or 0.01% of the total concentration of antibodies comprised in a composition of the invention are antibody fragments. Conversely in one embodiment at least 95%, 96%, 97%, 98%, 99%, 99.5%, 99.9%, 99.99% or 100% (suitably 100%) of the total concentration of antibodies comprised in the composition are intact antibodies. In one embodiment a polyclonal antibody may be purified and/or isolated from contaminating antibody fragments.

Advantageously, the intact antibodies of the invention (preferably blood-derived ovine or equine antibodies) demonstrate improved TNFα binding and/or neutralisation when compared to antibody fragments, as demonstrated by an improved TNFα binding capability (Examples 3 and 7) and/or neutralisation capability (Examples 4 and 8). Moreover, the intact antibodies of the invention are much less expensive to produce than antibody fragments which require additional processing and/or purification steps. Additionally, it is believed that ovine or equine antibodies demonstrate improved TNFα binding and/or neutralisation than antibodies from non-ovine or non-equine sources, such as intact bovine polyclonal antibodies.

The term “blood-derived” as used herein means that the antibodies are obtained from the blood of a host (e.g. non-human mammal) used to produce said antibodies. Typically blood-derived antibodies may be obtained by administering human TNFα or a fragment thereof to said host (e.g. non-human mammal) subcutaneously, intramuscularly, intraperitoneally, and/or intravenously.

A blood sample obtained from the non-human mammal (e.g. ovine) host may be further processed, e.g. to obtain serum or blood plasma. The antibody may therefore be obtainable from blood serum or blood plasma. Preferably, where the non-human mammal is an ovine host, the antibodies may be obtainable from blood serum. Preferably, where the non-human mammal is an equine host, the antibodies may be obtainable from blood plasma.

In one embodiment a blood sample (e.g. serum) comprises at least 1, 2, 3, 4, 5, 6, 7, or 8 g/L of antibodies that bind to (and preferably neutralise) human TNFα, preferably at least 3 or at least 5 g/L of antibodies that bind to (and preferably neutralise) human TNFα.

In one embodiment a blood sample (e.g. serum) comprises about 1 to about 12 g/L of antibodies that bind to (and preferably neutralise) human TNFα, for example about 3 to about 9 g/L of antibodies that bind to (and preferably neutralise) human TNFα.

In one embodiment a blood sample comprises antibodies that bind to human TNFα with an avidity of at least 1×10⁹ L/mol, preferably at least 1×10¹⁰ L/mol.

Preferably the non-human mammal is an ovine non-human mammal.

Ovine antibodies are antibodies that have been raised in a sheep. A number of advantages are associated with using sheep as production hosts for blood-derived antibodies that bind to human TNFα. The inventors have found that the concentration of antibodies that bind to human TNFα present in the sheep blood (e.g. serum) remains substantially constant over time provided the ovine continues to be immunised regularly (e.g. every 28 days). If immunisation is stopped it typically takes around 6 months from obtaining a maximum concentration of specific antibodies for the concentration to halve. Additionally, the substantially constant concentration of specific antibodies allows a high yield of blood-derived antibodies to be obtained per annum. In contrast, the inventors have found that the antibody yield per annum is far lower when using milk (e.g. bovine milk), eggs, or colostrum. Moreover, high concentrations of specific antibodies can be obtained at virtually any time point in the immunisation schedule (once maximum antibody concentrations have been reached). This is in contrast to other non-human mammals having fluctuating concentrations of specific antibodies in the blood, thus necessitating: measurement of the antibody concentration, and ensuring that a blood sample is obtained only when specific antibody concentrations are high. Therefore, using sheep as production hosts removes this additional step and/or removes unpredictability in the manufacturing method.

In addition, the present inventors have shown that specific blood-derived antibody concentrations between sheep are consistent.

Moreover, sheep are plentiful in the developed world, and easy to work with when compared to other non-human mammals.

Thus in one embodiment, a non-human mammal is a non-human mammal (e.g. ovine) that has a substantially constant blood concentration of polyclonal antibodies that bind to human TNFα after said non-human mammal has been administered an immunogen comprising human TNFα or a fragment thereof.

The term “substantially constant” as used in this context means that the blood concentration of polyclonal antibodies that bind to human TNFα decreases by 75% or less (preferably by 70%, 65%, or 60% or less, more preferably by 55% or less (e.g. 50% or less)) of the maximum blood concentration of said antibodies (100%) within and/or by 6 months (preferably within and by 6 months) after the maximum blood concentration of said antibodies has been reached.

Alternatively or additionally, the term “substantially constant” as used in this context may mean that within and/or by 6 months (preferably within and by 6 months) after the maximum blood concentration of polyclonal antibodies that bind to human TNFα has been reached (100%), the blood concentration of said polyclonal antibodies is at least 20% of the maximum, preferably at least 25%, 30%, 35%, or 40% or more preferably at least 45% (e.g. about 50%).

In one embodiment maximum blood concentration of polyclonal antibodies that bind to human TNFα occurs at at least 10 weeks from immunisation, such as at least 11 weeks from immunisation. Preferably the maximum blood concentration of polyclonal antibodies that bind to human TNFα occurs at about 12 weeks from immunisation.

Thus, the present invention includes a method for producing ovine antibodies for use in a composition of the invention, said method typically comprising:
i. administering an immunogen comprising a human TNFα or a fragment thereof to a sheep;
ii. allowing sufficient time for the generation of antibodies in the sheep; and
iii. obtaining the antibodies from the sheep.

The term “sheep” as used herein is synonymous with the term “ovine”. As used herein, sheep comprise any species that fall within the Ovis genus (e.g. Ovis ammon, Ovis orientalis aries, Ovis orientalis orientalis, Ovis orientalis vignei, Ovis Canadensis, Ovis dalli, Ovis nivicola).

The term “ovine antibody” as used herein is an antibody that has at least 85%, 90%, 95%, or 99% amino acid sequence identity to an antibody that has been raised in a sheep. Preferably an “ovine antibody” as used herein is an antibody that has 100% amino acid sequence identity to an antibody that has been raised in a sheep.

In one embodiment a composition of the present invention comprises only ovine antibodies, and thus excludes antibodies from a non-ovine source.

The antibody is typically obtainable from the sheep serum. Thus, methods for producing ovine antibodies described herein generate sheep antisera comprising antibodies capable of binding to and/or neutralising human TNFα. In one embodiment an antibody is isolated and/or purified, for example isolated and/or purified from a sheep antiserum.

Preferably the non-human mammal is an equine non-human mammal.

Equine antibodies are antibodies which have been raised in a horse. Advantageously, a high yield of blood-derived antibodies that bind to human TNFα can be obtained per annum by using horses as production hosts. Moreover, equine blood cells have been found to settle rapidly upon collection of a sample, thus avoiding the need for a time-consuming centrifugation step when obtaining plasma.

In embodiments where the non-human mammal is an equine non-human mammal, the manufacturing method may comprise obtaining blood plasma from a blood sample and returning the blood cells from said sample to said equine non-human mammal. Suitably, the blood cells may be returned in less than 24 hours, less than 12 hours, less than 6 hours, less than 1 hour or less than 30 minutes after obtaining the blood sample.

Thus, the present invention includes a method for producing equine antibodies for use in a composition of the invention, said method typically comprising:
i. administering an immunogen comprising a human TNFα or a fragment thereof to an equine;
ii. allowing sufficient time for the generation of antibodies in the equine; and
iii. obtaining the antibodies from the equine.

The term “horse” as used herein is synonymous with the term “equine”. As used herein, horses comprise any species that fall within the Equus genus. Preferably a horse is one or more from the species Equus ferus, such as Equus ferus caballus.

The term “equine antibody” as used herein is an antibody that has at least 85%, 90%, 95%, or 99% amino acid sequence identity to an antibody that has been raised in a horse. Preferably an “equine antibody” as used herein is an antibody that has 100% amino acid sequence identity to an antibody that has been raised in a horse.

In one embodiment a composition of the present invention comprises only equine antibodies, and thus excludes antibodies from a non-equine source.

The antibody is typically obtainable from the horse blood plasma. Thus, methods for producing equine antibodies described herein generate equine blood plasma comprising antibodies capable of binding to and/or neutralising human TNFα. In one embodiment an antibody is isolated and/or purified, for example isolated and/or purified from an equine blood plasma.

The immunogen used to generate an antibody of the present invention is a human TNFα, which has optionally been purified. The term “human TNFα” as used herein encompasses a full-length human TNFα, a variant thereof or a fragment thereof. Preferably the term “human TNFα” means a full-length human TNFα. Suitably, the human TNFα may be a recombinant human TNFα.

An immunogen may be a human TNFα comprising (or consisting of) SEQ ID No. 1. In one embodiment an immunogen is a fragment of SEQ ID No. 1. In one embodiment an immunogen is a human TNFα variant (or fragment thereof) having at least 70% (suitably at least 80%) sequence identity to SEQ ID No. 1. Suitably an immunogen is a human TNFα variant (or fragment thereof) having at least 90% (suitably at least 95%) sequence identity to SEQ ID No. 1.

An immunogen may be a human TNFα comprising (or consisting of) SEQ ID No. 2. In one embodiment an immunogen is a fragment of SEQ ID No. 2. In one embodiment an immunogen is a human TNFα variant (or fragment thereof) having at least 70% (suitably at least 80%) sequence identity to SEQ ID No. 2. Suitably an immunogen is a human TNFα variant (or fragment thereof) having at least 90% (suitably at least 95%) sequence identity to SEQ ID No. 2.

In one embodiment an immunogen comprises (or consists of) SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, or SEQ ID No. 13 or a sequence having at least 70% sequence identity thereto (for example at least 80% sequence identity thereto). In one embodiment an immunogen comprises (or consists of) a sequence having at least 90% (e.g. at least 95%) sequence identity to SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12 or SEQ ID No. 13. Preferably, an immunogen comprises (or consists of) SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12 or SEQ ID No. 13. In one embodiment an immunogen is a fragment or variant of one or more of said sequence(s).

In one embodiment a “variant” may be a mimic of the peptide or peptide fragment, which mimic reproduces at least one epitope of the peptide or peptide fragment. In another embodiment a “variant” may be a peptide or peptide fragment having at least one amino acid mutation or modification when compared to a sequence described herein. In one embodiment a variant is SEQ ID No. 13.

The “fragment” referred to herein may be a fragment of SEQ ID No. 1 having any number of amino acids from 1 to 156. Alternatively or additionally the “fragment” referred to herein may be a fragment of SEQ ID No. 2 having any number of amino acids from 1 to 232. The fragment preferably includes at least one epitope of human TNFα. The “fragment” may also have a common antigenic cross-reactivity and/or substantially the same in vivo biological activity as the human TNFα from which it is derived. For example, an antibody capable of binding to a fragment would also be capable of binding to the human TNFα from which it is derived. Alternatively, the fragment may share a common ability to induce a “recall response” of a T-lymphocyte which has been previously exposed to an antigenic component of human TNFα.

In one embodiment a fragment comprises (or consists of) SEQ ID No. 1, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, or SEQ ID No. 12 or a sequence having at least 70% sequence identity thereto (for example at least 80% sequence identity thereto). In one embodiment a fragment comprises (or consists of) a sequence having at least 90% (e.g. at least 95%) sequence identity to SEQ ID No. 1, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, or SEQ ID No. 12. Preferably, a fragment comprises (or consists of) SEQ ID No. 1, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, or SEQ ID No. 12.

In one embodiment an immunogen or fragment thereof comprises (or consists of) the N-terminal or N-terminal fragment of human TNFα (e.g. SEQ ID No. 1 or SEQ ID No. 2). In one embodiment the N-terminal or N-terminal fragment comprises (or consist of) SEQ ID No. 3, SEQ ID No. 4, SEQ ID No. 5, SEQ ID No. 6 or SEQ ID No. 7 or a sequence having at least 70% sequence identity thereto (for example at least 80% sequence identity thereto). In one embodiment the N-terminal or N-terminal fragment comprises (or consists of) a sequence having at least 90% (e.g. at least 95%) sequence identity to SEQ ID No. 3, SEQ ID No. 4, SEQ ID No. 5, SEQ ID No. 6 or SEQ ID No. 7. Preferably, the N-terminal or N-terminal fragment comprises (or consists of) SEQ ID No. 3, SEQ ID No. 4, SEQ ID No. 5, SEQ ID No. 6 or SEQ ID No. 7.

The polyclonal antibodies of the invention may exhibit cross-reactivity to, and/or neutralisation of, murine TNFα. This is highly surprising, as alternative/conventional TNFα antibodies such as Infliximab are not cross-reactive to, and/or do not neutralise, murine TNFα. Thus, the polyclonal antibodies of the invention demonstrate improved properties when compared to Infliximab.

Advantageously, antibodies binding to N-terminal fragments of human TNFα may exhibit improved neutralisation properties.

Without wishing to be bound by theory, it is believed that human TNFα comprises (or consists of) a plurality of epitopes. For example a human TNFα monomer may comprise at least 2 or 3 epitopes. Human TNFα is also believed to adopt a trimeric structure. Thus, a human TNFα trimer may comprise further epitopes. Without wishing to be bound by theory, it is believed that at least 5 to 15 antibodies (e.g. 10 to 15 antibodies) of the composition may bind to a human TNFα trimer. Suitably about 12 antibodies of the composition may bind to a human TNFα trimer.

The antibody composition of the invention comprises polyclonal antibodies, thus preferably said antibody composition comprises a population of antibodies wherein the population is capable of binding to multiple epitopes (preferably all epitopes) of human TNFα.

In one embodiment an antibody composition of the invention comprises a first antibody that binds to a first epitope of human TNFα and a second antibody that binds to a second epitope of human TNFα. Preferably, an antibody composition of the invention comprises a third antibody that binds to a third epitope of human TNFα. Suitably, an antibody composition of the invention may comprise further antibodies, each of which bind to different further epitopes of human TNFα.

An antibody composition of the invention suitably comprises antibodies that bind to SEQ ID No. 1 and/or SEQ ID No. 2. Suitably an antibody composition of the invention may comprise antibodies that bind to SEQ ID No. 13.

In one embodiment an antibody composition comprises antibodies that bind to one or more of (e.g. a plurality of) SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12 or SEQ ID No. 13, or a sequence having at least 70% sequence identity thereto (for example at least 80% sequence identity thereto). In one embodiment an antibody composition comprises antibodies that bind to one or more (e.g. a plurality of) sequence(s) having at least 90% (e.g. at least 95%) sequence identity to SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12 or SEQ ID No. 13. Preferably, an antibody composition comprises antibodies that bind to one or more (e.g. a plurality of) sequence(s) shown as SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12 or SEQ ID No. 13.

In one embodiment an antibody composition comprises antibodies that bind to the N-terminal of human TNFα (e.g. SEQ ID No. 1 or SEQ ID No. 2). In one embodiment said antibodies bind to one or more of (e.g. a plurality of) SEQ ID No. 3, SEQ ID No. 4, SEQ ID No. 5, SEQ ID No. 6 or SEQ ID No. 7 or a sequence having at least 70% sequence identity thereto (for example at least 80% sequence identity thereto). In one embodiment said antibodies bind to one or more (e.g. a plurality of) sequence(s) having at least 90% (e.g. at least 95%) sequence identity to SEQ ID No. 3, SEQ ID No. 4, SEQ ID No. 5, SEQ ID No. 6 or SEQ ID No. 7. Preferably, said antibodies bind to one or more (e.g. a plurality of) sequence(s) shown as SEQ ID No. 3, SEQ ID No. 4, SEQ ID No. 5, SEQ ID No. 6 or SEQ ID No. 7.

A plurality of sequences means at least 2 (e.g. at least 3, 4, 5, 6, 7, 8, 9, 10, 11 or 12) of said sequences. Suitably the term plurality of sequences means all of the recited sequences.

Antigens may be formulated with an adjuvant. Suitable adjuvants may include alum (aluminium phosphate or aluminium hydroxide), saponin (and its purified component Quil A), Freund's complete and incomplete adjuvant, RIBBI adjuvant, and other adjuvants used in research and veterinary applications.

The invention contemplates a wide variety of immunisation schedules. In one embodiment, a non-human mammal (e.g. a sheep or horse) is administered an immunogen on day zero and subsequently receives an immunogen at intervals thereafter (e.g. every 20-35 days, such as every 28 days). The interval range and dosage range required can be determined by the person skilled in the art based on inter alia the precise nature of the immunogen, the route of administration, and the nature of the formulation. Variations in these dosage levels can be adjusted using standard empirical optimisation routines. Similarly, it is not intended that the present invention be limited to any particular schedule for antibody collection. The collection time may be typically after day 56. Levels of the specific antibody, i.e. that which binds to the immunogen, may represent at least 2 g per litre of blood, serum or plasma (e.g. at least 3 g per litre of blood, serum or plasma).

The antibodies obtained from the non-human mammal (e.g. sheep or horse) may be subsequently purified thus providing a “purified fraction” as referred to herein. In one embodiment the antibodies may be purified by precipitation, chromatography, filtration, or combinations thereof. The purification method chosen may be one that enables IgG to remain in solution, co-isolate albumin, or combinations thereof.

The precipitation may be a sodium sulphate precipitation, or a caprylic acid precipitation. Most preferably, the precipitation is a caprylic acid precipitation. Said precipitation precipitates out contaminants, while the polyclonal antibody of the invention remains in solution. The amount of caprylic acid to be employed may be determined experimentally, however, in one embodiment 4-8% v/v (preferably 6% v/v) caprylic acid may be employed.

A composition of the invention may be filtered or may have been filtered. Preferably, a filtration comprises the use of a glass microfiber filter and a 0.2 um filter. Preferably, a filtration comprises the use of a 0.45 um filter and a 0.2 um filter. The composition may optionally be subjected to a further 0.2 um filtration step. The 0.45 um filter may be a glass microfiber filter having 0.45 um pore sizes, such as one commercially available from Millipore.

In one embodiment, a composition of the invention is a composition that has been subjected to at least a precipitation step and filtration. Preferably, a composition of the invention is a composition that has been subjected to a caprylic acid precipitation step and filtration, wherein the filtration comprises the use of a 0.2 um filter (e.g. the use of a glass microfiber (preferably 0.45 um filter) and a 0.2 um filter). More preferably, a composition of the invention is a blood plasma or blood serum that has been subjected to a caprylic acid precipitation step and filtration, wherein the filtration comprises the use of a 0.2 um filter (e.g. the use of a glass microfiber (preferably 0.45 um filter) and a 0.2 um filter).

It is particularly preferred in the present invention that the antibodies are not subjected to extensive purification, thereby reducing costs associated with manufacturing.

In a particularly preferable embodiment, a polyclonal antibody of the invention (or composition comprising the same) has not been affinity purified using TNFα (e.g. human TNFα).

Preferably, blood serum or blood plasma comprising a polyclonal antibody of the invention has not been affinity purified using TNFα (e.g. human TNFα).

In another embodiment a polyclonal antibody of the invention (or composition comprising the same) has not been subjected to a purification step. In one embodiment blood serum or blood plasma comprising a polyclonal antibody of the invention has not been purified.

In a more preferable embodiment a polyclonal antibody of the invention has been subjected to one or more precipitation step(s) (and optionally filtration) only, such as one precipitation step and optionally filtration only. For example, a polyclonal antibody of the invention may have been subjected to a caprylic acid precipitation step and filtration comprising the use of a 0.2 um filter (e.g. the use of a glass microfiber (preferably 0.45 um filter) and a 0.2 um filter) only.

Most preferably, blood serum (e.g. from an ovine) or blood plasma (e.g. from an equine) comprising a polyclonal antibody of the invention has been subjected to one or more precipitation step(s) and filtration only, such as one precipitation step and filtration only. For example, a blood serum or blood plasma comprising a polyclonal antibody of the invention may have been subjected to a caprylic acid precipitation step and filtration comprising the use of a 0.2 um filter (e.g. the use of a glass microfiber (preferably 0.45 um filter) and a 0.2 um filter) only.

In one embodiment a composition of the invention (preferably a composition that has not been affinity purified) binds to human TNFα with an EC₅₀ (binding titre) value of 15 mg/L or less. In one embodiment a composition of the invention (preferably a composition that has not been affinity purified) binds to human TNFα with an EC₅₀ (binding titre) value of 10 mg/L or less, 5 mg/L or less or 2.5 mg/L or less. Preferably, a composition of the invention (preferably a composition that has not been affinity purified) binds to human TNFα with an EC₅₀ (binding titre) value of 5 mg/L or less. More preferably, a composition of the invention (preferably a composition that has not been affinity purified) binds to human TNFα with an EC₅₀ (binding titre) value of 1 mg/L or less, such as about 0.77 mg/L. Said EC₅₀ value can be determined by ELISA in accordance with Example 7. The EC₅₀ value in this context may refer to the concentration of antibody/composition required to generate a half-maximal response.

In one embodiment a composition of the invention (preferably a composition that has not been affinity purified) achieves at least 70% cell survival in a cell survival assay. In one embodiment a composition of the invention (preferably a composition that has not been affinity purified) achieves at least 75%, 80% or 85% cell survival in a cell survival assay. Preferably, a composition of the invention (preferably a composition that has not been affinity purified) achieves at least 90%, more preferably at least 95% (e.g. 99% or 100%) cell survival in a cell survival assay. In one embodiment a composition of the invention (preferably a composition that has not been affinity purified) neutralises human TNFα cytotoxicity in a cell survival assay with an EC₅₀ value of 15 ug/ml or less. In one embodiment a composition of the invention (preferably a composition that has not been affinity purified) neutralises human TNFα cytotoxicity in a cell survival assay with an EC₅₀ value of 10 ug/ml or less, 5 ug/ml or less or 2.5 ug/ml or less. Preferably, a composition of the invention (preferably a composition that has not been affinity purified) neutralises human TNFα cytotoxicity in a cell survival assay with an EC₅₀ value of 1 ug/ml or less, more preferably 0.5 ug/ml or less (e.g. about 0.455 ug/ml). Said cell survival assay may be a standard in vitro L929 assay, preferably carried out as described in Example 8. The EC₅₀ value in this context refers to the concentration of antibody/composition required to protect 50% of the cell monolayer from human TNFα.

The intact polyclonal antibody of the invention may be formulated with a buffer. The buffer may include physiological salts such as sodium citrate and/or citric acid. A physiological salt may be present in the buffer at a concentration of 1-200 mM. Suitably a physiological salt may be present in a buffer at a concentration of approximately 5-50 mM (preferably at about 20 mM).

In one embodiment an antibody composition (e.g. medicament) is formulated as a solid. Preferably an antibody composition is formulated such that it is solid at ambient temperature and a liquid at body temperature, thus allowing release of the antibody when administered (e.g. rectally).

The antibody composition (e.g. medicament) is preferably formulated for rectal administration (via the anus). For example the antibody composition may be formulated as a suppository or an enema.

Most preferably the composition is formulated as a suppository. Suppositories can be prepared by mixing the an antibody of the invention with suitable excipients or carriers such as gelatin, cocoa butter, polyethylene glycol or a suppository wax. Said excipients or carriers are solid at ambient temperature but melt at body temperature (e.g. when rectally administered) allowing release of the antibody. It is preferred that suppositories (e.g. comprising gelatin) are not further coated, e.g. enteric coated. Thus, in one embodiment a suppository may comprise (or consist of) a polyclonal antibody of the invention (optionally in a suitable buffer) and an excipients or carrier selected from gelatin, cocoa butter, polyethylene glycol and a suppository wax. In one embodiment, an excipient or carrier consists of gelatin, cocoa butter, polyethylene glycol or a suppository wax (preferably gelatin).

The intact polyclonal antibody may be present at a concentration of 10-1000 mg. In one embodiment the intact polyclonal antibody is present at a concentration of 10-500 mg, such as 10-250 mg. Preferably antibodies may be present at a concentration of about 50-100 mg. The concentrations referred to may be the concentrations of total intact polyclonal antibodies, suitably total intact polyclonal IgG (i.e. including antibodies that do, as well as do not, bind to TNFα), e.g. at least 10% of said antibodies may bind to and neutralise TNFα. In one embodiment the foregoing embodiments refer to intact polyclonal antibodies that bind to (and preferably neutralise) TNFα.

In one embodiment, a composition of the invention comprises (or consists of):

| Ingredient | Concentration | Function |
|---|---|---|
| Total IgG (including intact | 0.2-20% w/w | Active ingredient |
| blood-derived anti-TNFα | | |
| Total IgG) | | |
| Excipient or carrier | To final weight | |

In one embodiment a composition of the invention comprises 0.2-10% w/w of intact polyclonal antibody, such as 1-7.5% w/w of intact polyclonal antibody. Preferably a composition of the invention comprises 1-2% w/w of intact polyclonal antibody. Alternatively, a composition of the invention preferably comprises 2-6% w/w of intact polyclonal antibody.

The total weight of the solid formulation may be at least 1 g, 2 g, 3 g or 4 g. For administration to an adult patient, said solid formulation (e.g. suppository) may be 4 g or greater. For administration to a pediatric patient, said solid formulation (e.g. suppository) may be less than 4 g, such as 2 g or less.

The intact polyclonal antibody may be present in a buffer, wherein the concentration of said buffer is 1-100 mM, such as 10-30 mM. A suitable buffer may be a citrate (e.g. sodium citrate saline) buffer.

In a particularly preferred embodiment, a composition of the invention comprises (or consists of):

| Ingredient | Concentration | Function |
|---|---|---|
| Total IgG (including intact | 50-100 mg | Active ingredient |
| blood-derived anti-TNFα | per suppository | |
| Total IgG) (e.g. in 20 mM | | |
| sodium citrate saline | | |
| (SCS), pH 6.0) | | |
| Gelatin | Up to 4 g | |

Where a composition of the invention is to be administered to a pediatric patient, the composition may comprise (or consist of):

| Ingredient | Concentration | Function |
|---|---|---|
| Total IgG (including intact | 50-100 mg | Active ingredient |
| blood-derived anti-TNFα | per suppository | |
| Total IgG) (e.g. in 20 mM | | |
| sodium citrate saline | | |
| (SCS), pH 6.0) | | |
| Gelatin | Up to 2 g | |

Alternatively, where a composition of the invention is to be administered to a pediatric patient, the composition may comprise (or consist of):

| Ingredient | Concentration | Function |
|---|---|---|
| Total IgG (including intact | 50-100 mg | Active ingredient |
| blood-derived anti-TNFα | per suppository | |
| Total IgG) (e.g. in 20 mM | | |
| sodium citrate saline | | |
| (SCS), pH 6.0) | | |
| Gelatin | Up to 1 g | |

In another embodiment, a composition of the invention comprises (or consists of):

| Ingredient | Concentration | Function |
|---|---|---|
| Total IgG (including intact | 50-100 mg | Active ingredient |
| blood-derived anti-TNFα | per suppository | |
| Total IgG) (e.g. in 20 mM | | |
| sodium citrate saline | | |
| (SCS), pH 6.0) | | |
| Gelatin | Up to 5 g | |

In one embodiment the composition is formulated as a liquid, more preferably as an enema. The composition may be formulated as part of a phosphate enema.

In some embodiments the liquid may include gas, such that the liquid composition is in (or is administered in) the form of a foam. Where the composition is in the form of a foam or is to be administered in the form of a foam, an antifoaming agent is preferably not present. Such compositions may be administered (e.g. rectally) to a subject by way of an aerosol.

In one aspect the invention provides a liquid composition (preferably an enema composition) for treating proctitis comprising (preferably consisting of):
a. intact blood-derived polyclonal antibodies that bind to a human tumour necrosis factor α (TNFα); and
b. one or more (preferably all) of: an antimicrobial, a suspension stability agent, an antifoaming agent, and a suspending agent;
   wherein the composition does not comprise means for protecting the antibodies during gastrointestinal transit.

The liquid or solid compositions/formulations described herein may be employed in any use or method of treatment described herein.

The intact polyclonal antibody may be present at a concentration of 1-100 g/L, such as 2-50 g/L. The intact polyclonal antibody may be present at a concentration of 5-100 g/L, such as 10-75 g/L. Preferably antibodies may be present at a concentration of about 20-75 g/L (e.g. 35-60 g/L). In one embodiment a composition may comprise at least about 20 (preferably at least 50 g/L) of intact polyclonal antibodies. The concentrations referred to may be the concentrations of total intact polyclonal antibodies, suitably total intact polyclonal IgG (i.e. including antibodies that do, as well as do not, bind to TNFα), e.g. at least 10% of said antibodies may bind to and neutralise TNFα. In one embodiment the foregoing embodiments refer to intact polyclonal antibodies that bind to (and preferably neutralise) TNFα.

A composition of the invention may comprise one or more antimicrobial. In one embodiment an antimicrobial is methylparaben (E218) and/or propylparaben (E216). Suitably, a composition of the invention comprises a combination of methylparaben (E218) and propylparaben (E216). An antimicrobial (e.g. each antimicrobial) may be present in the composition at a concentration of at least 0.2 g/L or at least 0.4 g/L. In one embodiment an antimicrobial is present in the composition at a concentration of at least 0.6 g/L or at least 1.0 g/L. Suitably, an antimicrobial may be present at a concentration of at least 1.5 g/L or at least 2.0 g/L. In some embodiments the antimicrobial is present at a concentration between 0.2 g/L to 1.0 g/L. In other embodiments the antimicrobial is present at a concentration between 1.0 g/L to 3.0 g/L (e.g. 1.5 g/L to 2.5 g/L).

A composition of the invention may comprise a suspension stability agent, for example glycine. The suspension stability agent (e.g. 200 mM glycine) may be present at a concentration of at least 5 g/L, or at least 10 g/L. In other embodiments the suspension stability agent (e.g. 200 mM glycine) is present at a concentration between 10 g/L to 20 g/L.

Alternatively or additionally, a composition of the invention may comprise an antifoaming agent, such as simethicone. Said antifoaming agent may be present at a concentration of at least 5 g/L, or at least 10 g/L. In one embodiment the antifoaming agent is present at a concentration of between 5 g/L and 25 g/L (suitably between 10 g/L and 20 g/L).

The composition of the invention may comprise a suspending agent, such as xantham gum. Said suspending agent may be present at a concentration of at least 1 g/L or 2 g/L. Suitably, said suspending agent may be present at a concentration between 1 g/L to 10 g/L, for example 3 g/L to 5 g/L.

In one embodiment a composition of the invention comprises (or consists of):

| Category | Material name | Concentration | |
|---|---|---|---|
| IgG | Total IgG (including intact | 25-100 | g/l |
| | blood-derived anti-TNFα | | |
| | Total IgG at ~10%) | | |
| Antimicrobial | Methylparaben (E218) | 0.5-5 | g/l |
| | Propylparaben (E216) | 0.1-1.2 | g/l |
| Suspension stability | Glycine (200 mM) | 5-25 | g/l |
| Antifoaming agent | Simethicone | 5-25 | g/l |
| Suspending agent | Xanthan gum | 1-10 | g/l |

In a particularly preferred embodiment, a composition of the invention comprises (or consists of):

| Category | Material name | Concentration | |
|---|---|---|---|
| IgG | Total IgG (including intact | 50 | g/l |
| | blood-derived anti-TNFα | | |
| | Total IgG at ~10%) | | |
| Antimicrobial | Methylparaben (E218) | 2 | g/l |
| | Propylparaben (E216) | 0.6 | g/l |
| Suspension stability | Glycine (200 mM) | 15 | g/l |
| Antifoaming agent | Simethicone | 16.89 | g/l |
| Suspending agent | Xanthan gum | 4 | g/l |

The compositions of the invention are preferably not formulated with a flavouring agent or sweetener, such as peppermint oil, vanilla essence, a sugar (e.g. glucose, sucrose, etc.), sugar alcohols, honey, fruit, syrups (e.g. maple syrup, rice syrup, birch syrup, pine syrup, hickory syrup, poplar syrup, palm syrup, sugar beet syrup, sorghum syrup, corn syrup, cane syrup, golden syrup, barley malt syrup, molasses (treacle), brown rice syrup, agave syrup, yacon syrup), acesulfame potassium (also known as Sunett), alitame (also known as aclame), aspartame (also known as Equal or Nutrasweet), anethole, cyclamate, glycyrrhizin, lo han guo, neotame, perillartine, saccharin (also known as Sweet ‘n’ Low), sodium saccharin, mannitol, stevioside, sucralose (also known as SucraPlus and Splenda), or inulin.

Preferably the antibodies of the present invention are not formulated with a means for protecting the antibodies during gastrointestinal transit. In other words, an external means for protecting the antibodies (i.e. not already part of e.g. a blood serum or blood plasma composition) is preferably not added. Preferably the antibodies are not administered (whether simultaneously or sequentially) with a means for protecting the antibodies during gastrointestinal transit. Such means may include one or more of: a protease inhibitor, an antacid, a post-translational modification of an antibody (e.g. PEGylation and/or glycosylation), a stabilizing amino acid modification, a lectin, egg (e.g. egg white) or a component thereof, a coating component (e.g. a coating matrix and/or enteric coating), a microbially-controlled delivery system, a polyacrylate, a means for encapsulation or entrapment of an antibody (e.g. microencapsulation and/or microspheres, such as albumin-chitosan mixed matrix microspheres), monomethoxypoly(ethylene) glycol (e.g. wherein the monomethoxypoly(ethylene) glycol is activated by cyanic chloride, succinimidyl succinate, and tresyl chloride), an emulsion, a colloidal polymer, and liposomes. A means for protecting the antibodies during gastrointestinal transit may be one or more described in WO 2018/138524 A1, which is incorporated herein by reference.

Preferably the antibodies of the invention are not encapsulated.

The term “disorder” as used herein also encompasses a “disease”. In one embodiment the disorder is a disease.

The term “proctitis” as used herein means inflammation of one or more (preferably all) of the anus, the rectum (e.g. the lining thereof), and the terminal 15 cm of the colon, or parts thereof. In one embodiment the term “proctitis” as used herein means inflammation of one or more of the anus, the rectum, and the terminal 10 or 5 cm of the colon, or parts thereof. In another embodiment the term “proctitis” as used herein means inflammation of the rectum and/or the anus, or parts thereof. Preferably the term “proctitis” as used herein means inflammation of the final 15 cm of the rectum (e.g. the final 10 cm of the rectum) and/or the anus, or parts thereof. Preferably the term “proctitis” as used herein means inflammation of the final 5 cm of the rectum and/or the anus, or parts thereof. In one embodiment the term “proctitis” means inflammation of the anus only or parts thereof.

Any cause for the inflammation is encompassed by the present invention. In one embodiment proctitis is one or more selected from: proctitis induced by chemotherapy, proctitis induced by radiotherapy, idiopathic proctitis, proctitis caused by a sexually transmitted disease, and proctitis caused by coeliac disease.

In one embodiment a subject for treatment in accordance with the invention has a sexually transmitted disease (e.g. gonorrhea, chlamydia, herpes and/or syphilis), coeliac disease or a combination thereof.

In one embodiment proctitis is one or more selected from: gonococcal proctitis (caused by gonorrhea), chlamydia proctitis (caused by chlamydia), herpes proctitis (caused by herpes simplex virus 1 or 2), and syphilitic proctitis (caused by syphilis).

In one embodiment a subject for treatment in accordance with the invention is undergoing chemotherapy, radiotherapy or a combination thereof.

The term “subject” as used herein refers to a mammal, such as a human or other animal. Preferably “subject” means a human subject.

Monoclonal antibody therapy is non-viable (e.g. due to high costs associated with production of monoclonal antibodies) for treatment of proctitis. Advantageously, a polyclonal antibody composition of the present invention provides a solution to the problem of treating said disorders. Said polyclonal antibody composition is comparatively inexpensive to manufacture, thus providing a viable therapeutic.

A suitable therapeutic is required to exhibit high specificity for human TNFα. Advantageously, the blood-derived polyclonal antibodies (e.g. ovine or equine) (owing to the method of manufacture) demonstrate high specificity for human TNFα when compared to non-blood derived (and/or e.g. non-ovine or non-equine) antibodies and/or monoclonal antibodies. Furthermore, by definition, a composition comprising monoclonal antibodies will only bind to a single epitope whereas a composition comprising polyclonal antibodies will bind to several, thereby increasing the efficacy with which the human TNFα is neutralised.

The term “treat” or “treating” as used herein encompasses prophylactic treatment (e.g. to prevent onset of a disease) as well as corrective treatment (treatment of a subject already suffering from a disease). Preferably “treat” or “treating” as used herein means corrective treatment.

The term “treat” or “treating” as used herein refers to the disorder and/or a symptom thereof.

Therefore a composition of the invention may be administered to a subject in a therapeutically effective amount or a prophylactically effective amount.

A “therapeutically effective amount” is any amount of the antibody, which when administered alone or in combination to a subject for treating proctitis (or a symptom thereof) is sufficient to effect such treatment of the disorder, or symptom thereof.

A “prophylactically effective amount” is any amount of the antibody that, when administered alone or in combination to a subject inhibits or delays the onset or reoccurrence of proctitis (or a symptom thereof). In some embodiments, the prophylactically effective amount prevents the onset or reoccurrence of proctitis entirely. “Inhibiting” the onset means either lessening the likelihood of the onset of proctitis (or symptom thereof), or preventing the onset entirely.

An appropriate dosage range is one that produces the desired therapeutic effect (e.g. wherein the antibody/composition is dosed in a therapeutically or prophylactically effective amount). A typical dosage regimen may comprise administering a composition of the invention at least four times per week. Preferably, a composition of the invention is administered daily.

The composition of the invention may be administered at least once, twice or three times per day. Preferably the composition is administered three times per day.

In a particularly preferred embodiment the composition is administered three times per day, seven days per week (i.e. daily). Administration is typically carried out until the proctitis or symptom thereof is no longer present, and/or until a subject's chemotherapy and/or radiotherapy regimen has been completed.

An appropriate single dose may be 1000 mg or less of polyclonal antibodies. In one embodiment a single dose is 500 mg or less, such as 250 mg or less of polyclonal antibodies. Preferably, the dose is 150 mg or less of polyclonal antibodies.

In one embodiment a single dose is at least 10 mg of polyclonal antibodies. In one embodiment a single does is at least 25 mg of polyclonal antibodies, preferably at least 50 mg of polyclonal antibodies.

In one embodiment a single dose is 10 mg to 1000 mg of polyclonal antibodies. In one embodiment a single dose is 10 mg to 500 mg of polyclonal antibodies, such as 10 mg to 250 mg of polyclonal antibodies. Preferably a single dose is 50 mg to 100 mg of polyclonal antibodies.

The foregoing single doses may be administered at least once, twice or three times per day. Preferably the composition is administered three times per day.

In a particularly preferred embodiment a single dose of 50 mg to 100 mg of polyclonal antibodies is administered twice daily (e.g. the daily dose may be 100 mg to 200 mg).

In a particularly preferred embodiment a single dose of 50 mg to 100 mg of polyclonal antibodies is administered three times daily (e.g. the daily dose may be 150 mg to 300 mg).

The above-referenced doses are particularly suitable for when the composition is comprised in a solid formulation (e.g. a suppository). Where the composition is comprised in a liquid (or foam) formulation (e.g. an enema) the dose may be higher. In one embodiment an appropriate single dose may be 10 g or less of polyclonal antibodies. In one embodiment a single dose is 5 g or less, such as 2.5 g or less of polyclonal antibodies.

In one embodiment a single dose is at least 0.05 g of polyclonal antibodies. In one embodiment a single dose is at least 0.10 g of polyclonal antibodies. In one embodiment a single dose is at least 0.25 g of polyclonal antibodies. In one embodiment a single does is at least 1 g of polyclonal antibodies, preferably at least 2 g of polyclonal antibodies.

In one embodiment a single dose is 0.05 g to 10 g of polyclonal antibodies. In one embodiment a single dose is 0.1 g to 10 g of polyclonal antibodies. In one embodiment a single dose is 0.1 g to 5 g of polyclonal antibodies. In one embodiment a single dose is 1 g to 10 g of polyclonal antibodies, such as 1 g to 7.5 g of polyclonal antibodies. Preferably a single dose is 2 g to 6 g of polyclonal antibodies. More preferably a single dose is 2.5 g to 5 g of polyclonal antibodies.

The foregoing single doses may be administered at least once, twice or three times per day. Preferably the composition is administered three times per day.

In a particularly preferred embodiment a single dose of 2 g to 6 g of polyclonal antibodies is administered twice daily (e.g. the daily dose may be 4 g to 12 g).

In a particularly preferred embodiment a single dose of 2 g to 6 g of polyclonal antibodies is administered three times daily (e.g. the daily dose may be 6 g to 18 g).

In a particularly preferred embodiment a single dose of 2.5 g or 5 g of polyclonal antibodies is administered twice daily (e.g. the daily dose may be 5 g or 10 g, respectively).

In a particularly preferred embodiment a single dose of 2.5 g or 5 g of polyclonal antibodies is administered three times daily (e.g. the daily dose may be 7.5 g or 15 g, respectively).

Where the composition is formulated as a liquid (e.g. enema) a single dose of at least 25 ml, 50 ml or 100 ml may be administered to a subject.

Suitably, the doses above refer to the total amount of intact polyclonal antibodies comprised in a composition of the invention, suitably total intact polyclonal IgG (i.e. including antibodies that bind to TNFα and antibodies that do not bind to TNFα). The intact polyclonal antibodies may be obtainable directly from a blood sample (e.g. antisera) or a purified fraction thereof. Preferably, the intact polyclonal antibodies have been purified from a blood sample (e.g. an ovine blood sample), such as antisera. For example, the sample may have been subjected to precipitation (preferably sodium sulphate or caprylic acid precipitation, most preferably caprylic acid precipitation), and filtration. Suitably, at least 5% (e.g. at least 10%) of the total intact polyclonal antibodies comprised in the composition bind to TNFα. More preferably at least 15% or at least 20% of the total intact polyclonal antibodies comprised in the composition bind to TNFα.

It is surprising that a therapeutic/prophylactic effect is observed at such low doses of total intact blood-derived polyclonal antibody, and is demonstrative of the high concentration of specific antibody (i.e. that binds to TNFα) produced when compared to non-blood-derived source, such as milk. It is furthermore surprising that a therapeutic/prophylactic effect is observed at such low doses of total intact ovine or equine (preferably ovine) polyclonal antibody, and is demonstrative of the high concentration of specific antibody (i.e. that binds to TNFα) produced using an ovine or equine (preferably ovine) host.

In some embodiments an antibody composition of the invention may be administered to a subject in combination with one or more further therapeutic(s). In one embodiment the antibody composition is administered while a subject is undergoing chemotherapy. One or more further therapeutic(s) may be administered sequentially or simultaneously with an antibody composition of the invention.

In one embodiment an antibody composition is administered in combination with a therapeutic that treats proctitis. In one embodiment a therapeutic may be an aminosalicylate (5-ASA), a corticosteroid (e.g. prednisone or budesonide (Entocort® EC)), mesalazine (e.g. Asacol) or an antibiotic. In one embodiment an antibody composition is administered as part of a foam enema in combination with Asacol.

Embodiments related to the various antibody compositions for use of the invention are intended to be applied equally to the uses, methods, solid or liquid compositions, and vice versa.

### Sequence Comparison

For sequence comparison, typically one sequence acts as a reference sequence, to which test sequences may be compared. When using a sequence comparison algorithm, test and reference sequences are input into a computer, subsequent coordinates are designated, if necessary, and sequence algorithm program parameters are designated. The sequence comparison algorithm then calculates the percentage sequence identity for the test sequence(s) relative to the reference sequence, based on the designated program parameters.

Optimal alignment of sequences for comparison may be conducted, for example, by the local homology alignment algorithm of Smith and Waterman [Adv. Appl. Math. 2: 484 (1981)], by the algorithm of Needleman & Wunsch [J. Mol. Biol. 48: 443 (1970)] by the search for similarity method of Pearson & Lipman [Proc. Nat'l. Acad. Sci. USA 85: 2444 (1988)], by computer implementations of these algorithms (GAP, BESTFIT, FASTA, and TFASTA—Sequence Analysis Software Package of the Genetics Computer Group, University of Wisconsin Biotechnology Center, 1710 University Avenue, Madison, Wis. 53705), or by visual inspection [see Current Protocols in Molecular Biology, F. M. Ausbel et al, eds, Current Protocols, a joint venture between Greene Publishing Associates, In. And John Wiley & Sons, Inc. (1995 Supplement) Ausbubel].

Examples of algorithms suitable for determining percent sequence similarity are the BLAST and BLAST 2.0 algorithms [see Altschul (1990) J. Mol. Biol. 215: pp. 403-410; and “http://www.ncbi.nlm.nih.gov/” of the National Center for Biotechnology Information].

In one homology comparison, the identity exists over a region of the sequences that is at least 10 or 20 or 30 or 40 or 50 amino acid residues in length. In another homology comparison, the identity exists over a region of the sequences that is at least 60 or 70 or 80 or 90 or 100 amino acid residues in length.

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

Conservative amino acid substitutions may include:

Basic: arginine
lysine
histidine
   Acidic: glutamic acid
aspartic acid
   Polar: glutamine
asparagine
   Hydrophobic: leucine
isoleucine
valine
   Aromatic: phenylalanine
tryptophan
tyrosine
   Small: glycine
alanine
serine
threonine
methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethyl homo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively. The headings provided herein are not limitations of the various aspects or embodiments of this disclosure. Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. It is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure. It must be noted that as used herein and in the appended claims, the singular forms “a”, “an”, and “the” include plural referents unless the context clearly dictates otherwise. Thus, for example, reference to “an antibody” includes a plurality of such candidate agents and reference to “the antibody” includes reference to one or more antibodies and equivalents thereof known to those skilled in the art, and so forth. The publications discussed herein are provided solely for their disclosure prior to the filing date of the present application. Nothing herein is to be construed as an admission that such publications constitute prior art to the claims appended hereto. The invention will now be described, by way of example only, with reference to the following Figures and Examples.

### FIGURES

Embodiments of the invention will now be described, by way of example only, with reference to the accompanying Figures, in which:

FIG. 1 shows results of a direct ELISA for detection of anti-TNFα IgG in antisera (•) and IgG purified by way of caprylic acid precipitation (▴).

FIG. 2 shows results of an immunocytotoxicity assay (ICTA) testing the neutralising activity of Anti-TNFα Fragment (▪), Intact Anti-TNFα (▴) and TNFα antisera (•) using L929 cells. Starting concentration: Anti-TNFα Fragment—50 mg/ml; Intact Anti-TNFα—210 mg/ml; TNFα antisera protein concentration—86 mg/ml.

FIG. 3 shows the results of an ELISA comparing binding to murine TNFα by ovine blood-derived intact PcAbs (anti-human TNFα IgG) and monoclonal antibody Infliximab. Anti-human TNFα IgG (•), Infliximab (▪), and negative control (PCB) (▴).

FIG. 4 shows the results of a second ELISA comparing binding to murine TNFα by ovine blood-derived intact PcAbs (anti-human TNFα IgG) and monoclonal antibody Infliximab. Anti-human TNFα IgG (•), Infliximab (▪), and negative control (PCB) (▴).

FIG. 5 shows the results of an immunocytotoxicity assay comparing cell survival in the presence of a 5 ng/ml challenging dose of murine TNFα when either the ovine blood-derived intact PcAbs (anti-human TNFα IgG) or monoclonal antibody Infliximab are administered. The results were obtained from 2× plates for the ovine blood-derived intact PcAbs (“Ovine IgG”=•+♦) and 2× plates for Infliximab (“Infliximab”=▪+hollow circle).

### EXAMPLES

### Example 1

### Preparation of Ovine Antisera to Human TNFα

Mature human TNFα (hTNFα) (UniProtKB Accession No.: P01375) was obtained from R&D Systems, Boehringer. The amino acid sequence is shown as SEQ ID No. 1.

The immunogen for the primary immunisation of merino weather sheep comprised Freund's complete adjuvant and 100 μg of hTNFα per sheep. The protein:adjuvant mixture was injected subcutaneously and equally into 6 injection sites chosen for their proximity to the axillary, inguinal, and prescapular drainage lymph glands. Each sheep was reimmunized at 28-day intervals with 100 μg of hTNFα and Freund's incomplete adjuvant, and blood samples were collected 14 days later at approximately 4 weekly intervals at processing facilities at Turretfield Research Centre (Rosedale, South Australia, Australia) according to strict state and national ethical guidelines for animal welfare. The animals were not terminally bled. A total of 10 mL of blood per kg of body weight can be collected from the external jugular vein without detriment to the animal.

Ovine antisera was subsequently stored at −20° C.

### Example 2

### Purification of Polyclonal Ovine Antibodies to Human TNFα (Intact Anti-TNFα)

Two different methods for purification of ovine polyclonal antibodies (PcAb) were employed. Either caprylic acid precipitation, which precipitates albumin and keeps the IgG in solution, or the sodium sulphate precipitation that precipitates IgG were used. The purified IgG was filtered and stored at −20° C. ready for inclusion in the proposed formulation for rectal administration or for further characterisation.

### Example 3

### Enzyme-Linked Immunosorbent Assay for Characterising Antibody Binding

The specific antibodies produced bound to multiple epitopes on the surface of recombinant human TNFα (rhTNFα) but not to recombinant rodent TNFα. The avidity of binding was extremely high.

A direct ELISA assay was developed for detection of anti-TNFα IgG in the ovine antisera and in the purified fraction of IgG (purified by way of caprylic acid precipitation) from this antisera (Intact Anti-TNFα). Immulon 4HBx microtiter plates were coated with 1 μg/mL hTNFα. Plates were washed with 3 changes of phosphate-buffered saline (PBS) containing 0.1% Tween 20 (PBST) and blocked for 1 hour at 37° C. with blocking buffer (2.5% fetal calf serum diluted in PBS). Plates were washed and incubated for 1 hour at 37° C. with antisera at initial dilutions of 1:1000, followed by 1:2 serial dilutions; washed with PBST; and incubated with a donkey anti-ovine IgG horseradish peroxidase conjugate for 1 hour at 37° C. After further washing, 3,3′,5,5′-Tetramethylbenzidine (TMB) liquid substrate solution was added, and the reaction was stopped after approximately 10 minutes by the addition of 1.0 M HCL before reading the optical density at 450 nm.

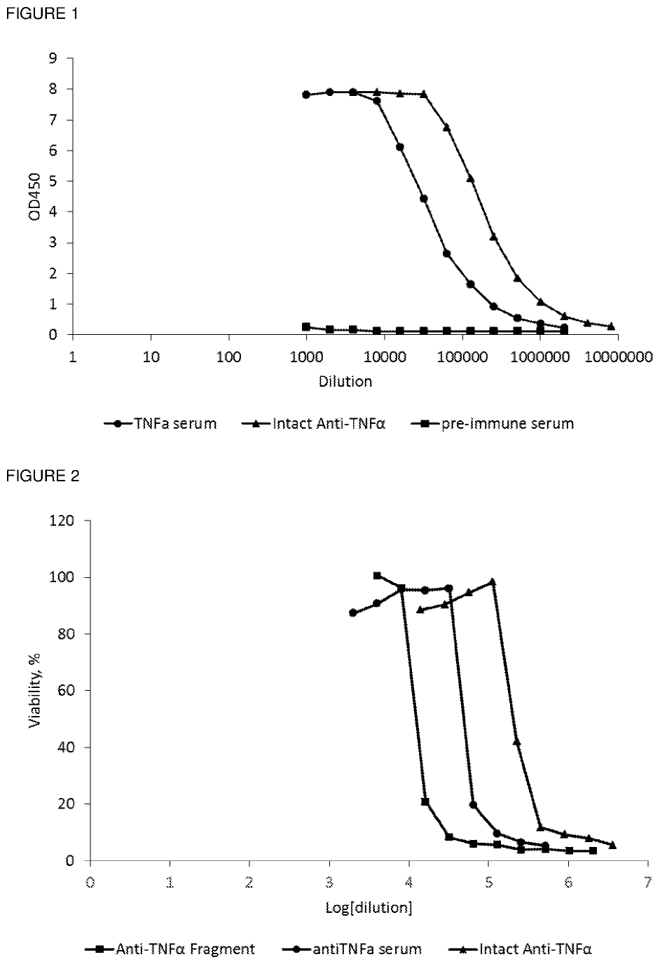

The developed assay showed very low background which was tested using pre-immune ovine serum (FIG. 1).

### Example 4

### Immunocytotoxicity Assay for Characterising Antibody Neutralisation

The L929 mouse fibrosarcoma cell line (commercially available from Sigma-Aldrich, The Old Brickyard, New Road, Gillingham, Dorset, SP84XT, UK) was used to test the cytotoxic effects of TNFα as well as the neutralising ability of antibodies to TNFα. An assay was therefore developed to test neutralisation of the cytotoxic effect of rhTNFα by the ovine PcAb in the antisera, and by purified IgG from the antisera (Intact Anti-TNFα), and by fragments thereof (Anti-TNFα Fragment).

Anti-TNFα Fragment was prepared by subjecting a portion of the stock of ovine antisera of Example 1 to papain digestion. The Fab were present at a concentration of 10 g/L and about 10% of the total Fab were specific for TNFα. An affinity chromatography step was not included in its manufacture. In the presence of excess of Fab, about 12 molecules of Fab become attached to each TNFα trimer.

As a challenging dose the IC90 hTNFα concentration of 13 ng/ml determined from a cytotoxicity assay (data not shown) was used. Briefly, L929 cells containing twice the necessary challenging dose in DMEM were co-incubated with an equal volume of various dilutions of hTNFα antisera or Anti-TNFα Fragment or Intact Anti-TNFα for 24 h. As a positive control (maximum killing), 2.5 μg/ml hTNFα was used. Antibody toxin neutralisation titres were estimated by colorimetric assays based on cell staining with neutral red.

Antibody toxin neutralisation titres were estimated by colorimetric assays based on cell staining with neutral red (representative curves shown in FIG. 2). Advantageously, Intact Anti-TNFα showed a significantly improved neutralisation ability when compared to Anti-TNFα Fragment.

The specific antibody concentration was calculated as follow:

Specific Ab conc[g/L]=[CCD(μg/L)−LC50(μg/L)]×[MW Ab/(MW Ag×BS)]×EC50×10⁻⁶
CCD (μg/L)—challenging dose=13 μg/L (LC90 determined from the TNFα cytotoxicity on L929 cells)
LC50 (μg/L)=0.3 μg/L (determined from the TNFα cytotoxicity on L929 cells)
BS—binding sites=2 for whole IgG
MW Ab=160000 Da
MW Ag (TNFα)=51000 Da

Taking the above into account, the specific PcAb concentration in the antisera was calculated at 2.9 g/L.

### Comparative Example 5

Comparative Analysis of Specific Antibody Titres from Sheep Serum (Ovine) & Hen Eggs

A study was undertaken to assess the concentration and avidities of specific IgΥ obtained from hen eggs in comparison with specific antibody concentrations from ovine antisera.

A group of 10 chickens and 5 sheep were immunised with human interleukin-6 (hIL-6, a pro-inflammatory cytokine like TNFα) and the titres and avidities of the resultant specific PcAb was compared. The average avidity constants were 1.3×10¹⁰ L/mol for chicken IgΥ vs 3.1×10¹⁰ L/mol for the ovine antibodies. However, the levels of specific PcAb attained in the sheep (with an average titre of 1:200,000) were more than ten times the titres found in egg yolk (≤1:20,000). This tenfold or more difference in the concentration of specific PcAb was also apparent when sheep and hens were immunised with a number of other immunogens.

The above experiment shows the advantages of antibodies derived from blood, and from an ovine source in particular.

### Example 6

Comparative Analysis of Specific Antibody Titres Sourced from Sheep Serum (Ovine) & Cow's Milk (Bovine)

A study was undertaken to assess the potential of colostrum and milk from suitably immunised cows as a source of PcAb.

Cows were immunised with human TNFα and the titres of the resulting specific PcAb determined first in the colostrum and then in serial samples of milk. The maximum titre obtained in colostrum was 1:275,000 (as compared with 1:800,000 in ovine antisera) and, after the first milking, levels rapidly fell to approximately 1:27,500.

Thus, blood-derived sources were shown to yield consistently higher concentrations of antibodies that bind to human TNFα when compared to milk/colostrum-derived sources.

### Example 7

### Binding of Ovine Blood-Derived Polyclonal Antibody to Human TNFα

The titre of the ovine blood-derived polyclonal anti-TNFα antibodies (Intact Anti-TNFα) were determined by indirect ELISA. Immulon^{R} 4 HBX flat bottom microfilter plates (Thermo Scientific) were coated with human recombinant TNFα at a concentration of 1 μg/mL (100 μl/well) using coating buffer (phosphate-buffered saline, PBS, containing 8 g/L NaCl, 0.2 g/L KCl, 1.44 g/L Na₂HPO₄, 0.24 g/L KH₂PO₄, pH 7.4) and incubated at 4° C. overnight. Plates were washed three times with PBS containing 0.1% Tween 20 (PBST) and blocked with 2.5% foetal bovine serum in PBS (150 μl/well) for 2 hours at 37° C. Plates were subsequently washed three times with PBST and incubated with the anti-TNFα polyclonal antibody composition (serum that had been purified using caprylic acid 6% v/v and filtered using a glass microfiber and 0.2 um filter) (100 μl/well) at appropriate dilutions. PolyCAb B (binds to C. difficile toxins) served as negative control. Wells were washed thrice with PBST and 100 μl of diluted (1:10,000) donkey anti-sheep antibody coupled to Horse Radish Peroxidase (Sigma) was added and incubated for 1 hour at 37° C. Then the plates were washed with PBST three times and 100 ml of substrate solution 3,3′,5,5′-tetramethylbenzidine (TMB) left for around 4 hours to reach room temperature was added. The plates were allowed to stand at room temperature for 5 minutes. The reaction was stopped by adding 50 ml/well of 1M HCl and plates were read in a PolarStar plate reader at 450 nm and 690 nm. All samples were tested in either duplicate or triplicate. The 50% binding titres were estimated using Graph Pad Prism 7.

The ovine blood-derived polyclonal antibodies were found to bind to human TNFα with an EC₅₀ value of 0.77 mg/L.

### Example 8

### Cell Survival Assay (Human TNFα)

The neutralising capacities of the blood-derived ovine polyclonal antibodies (Intact Anti-TNFα-serum that had been purified using caprylic acid 6% v/v and filtered using a glass microfiber and 0.2 um filter) and Infliximab (Schering-Plough Ltd) were assessed and quantified using a neutral red uptake (NRU) based assay. L929 cells were used which are known to undergo cell death when exposed to TNFα. The assay was performed using an indicator plate that was seeded at 7.5×10³/well (100 μL) with L929 cells and grown for approximately 24 h at 37° C. in a 5% CO₂ humidified atmosphere.

L929 cells were maintained using Dulbecco's modified Eagle's medium (DMEM) (Sigma) supplemented with 10% heat inactivated foetal calf serum (Sigma), 2 mM L-glutamine (Sigma) and 5% Penicillin/streptomycin (Sigma). Cells were maintained in 75 cm² flasks seeded weekly at a density of 2×10⁵ in 30 mL of culture medium. The cells were routinely maintained.

For each antibody sample, two-fold serial dilutions were performed using DMEM in a 96-well dilution plate, followed by the addition of an equal volume of media containing TNFα. A fixed concentration of TNFα was used (the challenging dose=12 ng/ml) and was based on the LC98 (Lethal Concentration to cause 98% cell death). This concentration was shown to be sufficient to cause close to 100% cell rounding. A TNFα cytotoxicity curve was included per plate as a control for inter assay variation, monitor antigen stability and reproducibility.

Antibody in DMEM acted as negative control, and TNFα challenging dose acted as positive control. The dilution plate was incubated for 1 hr at room temperature. Following incubation, 100 μL of sample from the dilution plate were transferred to the corresponding wells in the indicator plate containing 100 μl of media, thus making the total volume 200 μl. The indicator plate was then placed back in the incubator at 37° C., 5% CO₂. After 48 hours, the plates were washed three times with Phosphate buffered saline before 100 μl destain solution (50% ethanol and 1% acetic acid) added and placed on a plate rocker for 15 minutes. The plates were read in a PolarStar plate reader at a wavelength of 540 nm (test measurement) and 690 nm (background measurement). The absorbance for each well was calculated by subtracting the background measurement from the test measurement. Percentage cell death was calculated as follows:

$$Cell survival ( \% ) = \frac{( absorbance - positive control )}{negative control - positive control} \times 100$$

Results were obtained from a total of 6 plates performed on 3 separate days in duplicate (n=2). The ovine polyclonal antibodies conferred 100% cell survival in the assay at the highest concentrations used, compared to Infliximab, for which survival was less than 70%. Specifically, an average of 26.36% (range: 15.45-39.5%) difference was observed between the optimum survival of L929 cells treated with infliximab and the ovine polyclonal antibodies. This means that infliximab fails to completely neutralise hrTNFα.

The dilution of ovine polyclonal antibodies required to protect 50% of the cell monolayer was estimated using GraphPad Prism 7 giving an average EC₅₀ value of 0.455 ug/ml. This was surprising, as similar preparations of bovine colostrum-derived polyclonal antibodies have been reported to neutralise human TNFα with an EC₅₀ value of ˜16 ug/ml. Thus, the ovine blood-derived polyclonal antibodies are much improved when compared to the bovine polyclonal antibodies.

### Example 9

Comparison of Blood-Derived Polyclonal Antibodies with Monoclonal Antibodies

A comparative antigen-binding assay was performed using blood-derived ovine polyclonal antibodies that bind to human TNFα and Infliximab (Schering-Plough Ltd), a monoclonal antibody that binds to human TNFα.

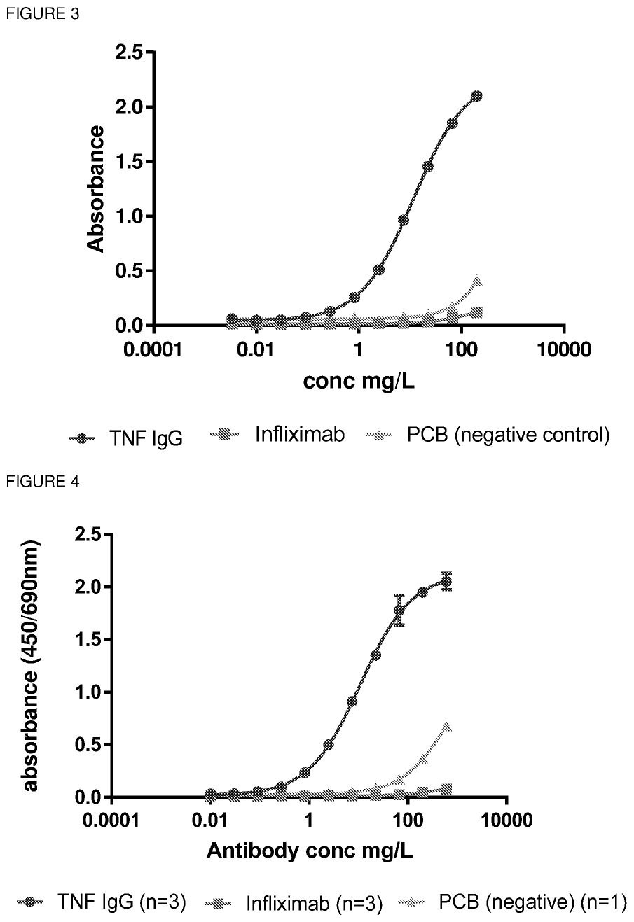

FIG. 3 shows that the monoclonal antibody, Infliximab, does not bind to murine TNFα, whereas the ovine derived polyclonal antibodies raised towards human TNFα do.

The blood-derived polyclonal antibodies of the invention were shown to bind to and neutralise murine TNFα albeit at a concentration approximately 100-fold higher than that needed to neutralise human TNFα. No neutralisation of murine TNFα was observed by Infliximab, indicative of an overall reduced neutralisation capability when compared to antibodies of the invention.

### Example 10

### Binding of Ovine Blood-Derived Polyclonal Antibody to Murine TNFα

The titres of Infliximab and blood-derived ovine polyclonal antibodies (Intact Anti-TNFα) were determined by indirect ELISA. Immulon^{R} 4 HBX flat bottom microfilter plates (Thermo Scientific) were coated with murine recombinant TNFα at a concentration of 1 μg/mL (100 μl/well) using coating buffer (phosphate-buffered saline, PBS, containing 8 g/L NaCl, 0.2 g/L KCl, 1.44 g/L Na₂HPO₄, 0.24 g/L KH₂PO₄, pH 7.4) and incubated at 4° C. overnight. Plates were washed three times with PBS containing 0.1% Tween 20 (PBST) and blocked with 2.5% foetal bovine serum in PBS (150 μl/well) for 2 hours at 37° C. Plates were subsequently washed three times with PBST and incubated with the anti-TNFα polyclonal antibody composition (serum that had been purified using caprylic acid 6% v/v and filtered using a glass microfiber and 0.2 um filter) or Infliximab (100 μl/well) at appropriate dilutions. PolyCAb B served as negative control. Wells were washed thrice with PBST and 100 μl of diluted (1:10,000) donkey anti-sheep antibody coupled to Horse Radish Peroxidase (Sigma) was added and incubated for 1 hour at 37° C. Then the plates were washed with PBST three times and 100 ml of substrate solution 3,3′,5,5′-tetramethylbenzidine (TMB) left for around 4 hours to reach room temperature was added. The plates were allowed to stand at room temperature for 5 minutes. The reaction was stopped by adding 50 ml/well of 1M HCl and plates were read in a PolarStar plate reader at 450 nm and 690 nm. All samples were tested in either duplicate or triplicate. The 50% binding titres were estimated using Graph Pad Prism 7.

FIG. 4 shows that the monoclonal antibody Infliximab does not bind to murine TNFα whereas the ovine derived polyclonal antibody raised towards human TNFα does with an average EC₅₀ of 11.35 mg/L (CV=5.5%). A total of 2 plates was performed in triplicate (n=3).

### Example 11

### Cell Survival Assay (Murine TNFα)

The neutralising capacities of the blood-derived ovine polyclonal antibodies (Intact Anti-TNFα-serum that had been purified using caprylic acid 6% v/v and filtered using a glass microfiber and 0.2 um filter) and Infliximab (Schering-Plough Ltd) were assessed and quantified using a neutral red uptake (NRU) based assay. L929 cells were used which are known to undergo cell death when exposed to TNFα. The assay was performed using an indicator plate that was seeded at 7.5×10³/well (100 μL) with L929 cells and grown for approximately 24 h at 37° C. in a 5% CO₂ humidified atmosphere.

L929 cells were maintained using Dulbecco's modified Eagle's medium (DMEM) (Sigma) supplemented with 10% heat inactivated foetal calf serum (Sigma), 2 mM L-glutamine (Sigma) and 5% Penicillin/streptomycin (Sigma). Cells were maintained in 75 cm² flasks seeded weekly at a density of 2×10⁵ in 30 mL of culture medium. The cells were routinely maintained.

For each antibody sample, two-fold serial dilutions were performed using DMEM in a 96-well dilution plate, followed by the addition of an equal volume of media containing TNFα. A fixed concentration of murine TNFα was used (the challenging dose=5 ng/ml) and was based on the LC98 (Lethal Concentration to cause 98% cell death). This concentration was shown to be sufficient to cause close to 100% cell rounding. A TNFα cytotoxicity curve was included per plate as a control for inter assay variation, monitor antigen stability and reproducibility.

Antibody in DMEM acted as negative control, and TNFα challenging dose acted as positive control. The dilution plate was incubated for 1 hr at room temperature. Following incubation, 100 μL of sample from the dilution plate were transferred to the corresponding wells in the indicator plate containing 100 μl of media, thus making the total volume 200 μl. The indicator plate was then placed back in the incubator at 37° C., 5% CO₂. After 48 hours, the plates were washed three times with Phosphate buffered saline before 100 μl destain solution (50% ethanol and 1% acetic acid) added and placed on a plate rocker for 15 minutes. The plates were read in a PolarStar plate reader at a wavelength of 540 nm (test measurement) and 690 nm (background measurement). The absorbance for each well was calculated by subtracting the background measurement from the test measurement. Percentage cell death was calculated as follows:

$$Cell survival ( \% ) = \frac{( absorbance - positive control )}{negative control - positive control} \times 100$$

The dilution required to protect 50% of the cell monolayer was estimated using GraphPad Prism 7.

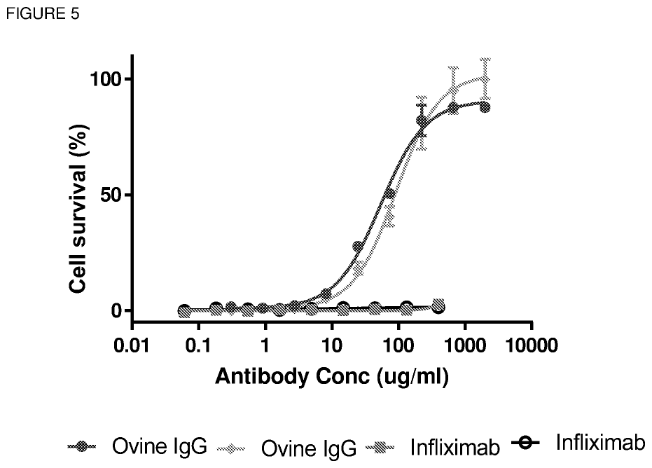

FIG. 5 shows that Infliximab confers no protection to cells exposed to murine TNFα. In contrast, for the ovine polyclonal antibodies an average EC₅₀ of 95.15 μg/ml (CV=25.6%) were observed and ˜100% survival observed at the higher doses. A total of 5 plates was performed on 3 separate days in duplicate (n=2).

### Example 12

### Formulation of a Composition for Treating Proctitis (Suppository)

The blood-derived polyclonal antibodies of the invention are formulated as a suppository as follows:

| Ingredient | Concentration | Function |
|---|---|---|
| Total IgG (including intact | 50-100 mg | Active ingredient |
| ovine anti-TNFα Total IgG | per suppository | |
| at ~10%) in 20 mM | | |
| SCS, pH 6.0 | | |
| Gelatin | Up to 4 g | |

### Example 13

### Formulation of a Composition for Treating Proctitis (Enema)

The blood-derived polyclonal antibodies of the invention are formulated as a liquid enema as follows:

| Category | Material name | Concentration | |
|---|---|---|---|
| IgG | Total IgG (including intact | 50 | g/l |
| | ovine anti-TNFα Total IgG | | |
| | at ~10%) | | |
| Antimicrobial | Methylparaben (E218) | 2 | g/l |
| | Propylparaben (E216) | 0.6 | g/l |
| Suspension stability | Glycine (200 mM) | 15 | g/l |
| Antifoaming agent | Simethicone | 16.89 | g/l |
| Suspending agent | Xanthan gum | 4 | g/l |

### Example 14

### Case Study

A 50 year old male is diagnosed proctitis. The subject is instructed by his physician to rectally administer a suppository of Example 12 twice per day. Treatment is continued until proctitis (or symptoms thereof) is no longer present.

### Example 15

### Case Study

A 33 year old female is diagnosed with proctitis. The subject is instructed by her physician to rectally administer a suppository of Example 12 three times per day. Once proctitis (or symptoms thereof) is no longer present the dosing frequency is reduced to twice daily.

### Example 16

### Case Study

A 67 year old female is diagnosed with proctitis. The subject is administered an enema of Example 13 (50 ml) three times per day. Once proctitis (or symptoms thereof) is controlled the enema is administered twice daily until proctitis (or symptoms thereof) is no longer present.

SEQUENCES
SEQ ID No. 1
VRSSSRTPSDKPVAHVVANPQAEGQLQWLNRRANALLANGVELRDNQLVVP
SEGLYLIYSQVLFKGQGCPSTHVLLTHTISRIAVSYQTKVNLLSAIKSPCQ
RETPEGAEAKPWYEPIYLGGVFQLEKGDRLSAEINRPDYLDFAESGQVYFG
IIAL
SEQ ID No. 2
MSTESMIRDVELAEEALPKKTGGPQGSRRCLFLSLFSFLIVAGATTLFCLL
HFGVIGPQREEFPRDLSLISPLAQAVRSSSRTPSDKPVAHVVANPQAEGQL
QWLNRRANALLANGVELRDNQLVVPSEGLYLIYSQVLFKGQGCPSTHVLLT
HTISRIAVSYQTKVNLLSAIKSPCQRETPEGAEAKPWYEPIYLGGVFQLEK
GDRLSAEINRPDYLDFAESGQVYFGIIAL
SEQ ID No. 3
MSTESMIRDVELAEEALPKKTGGPQGSRRCLFLSLFSFLIVAGATTLFCLL
HFGVIGPQREEFPRDLSLISPLAQAVRSSSRTPSDKPVAHVVANPQAEGQL
QWLNRRANALLANGVELRDNQLVVPSEGLYLIYSQVLFKGQGCP
SEQ ID No. 4
VRSSSRTPSDKPVAHVVANPQAEGQLQWLNRRANALLANGVELRDNQLVVP
SEGLYLIYSQVLFKGQGCP
SEQ ID No. 5
VRSSSRTP
SEQ ID No. 6
HVVANPQAEGQLQWLNRR
SEQ ID No. 7
NGVELR
SEQ ID No. 8
VPSEG
SEQ ID No. 9
CPSTHVL
SEQ ID No. 10
ISRIAVSYQTK
SEQ ID No. 11
PCQRETPEGAEAK
SEQ ID No. 12
DRLSAEINRPDYLDFA
(Variant Human TNFα P84L)
SEQ ID No. 13
MSTESMIRDVELAEEALPKKTGGPQGSRRCLFLSLFSFLIVAGATTLFCLL
HFGVIGPQREEFPRDLSLISPLAQAVRSSSRTLSDKPVAHVVANPQAEGQL
QWLNRRANALLANGVELRDNQLVVPSEGLYLIYSQVLFKGQGCPSTHVLLT
HTISRIAVSYQTKVNLLSAIKSPCQRETPEGAEAKPWYEPIYLGGVFQLEK
GDRLSAEINRPDYLDFAESGQVYFGIIAL

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. An antibody composition for use in treating proctitis, wherein the antibody composition comprises intact blood-derived polyclonal antibodies that bind to a human tumour necrosis factor α (TNFα).

2. Use of an antibody in the manufacture of a medicament for treating proctitis, wherein the antibody is an intact blood-derived polyclonal antibody that binds to a human tumour necrosis factor α (TNFα).

3. A method of treating proctitis, the method comprising administering an antibody composition to a subject, wherein the antibody composition comprises intact blood-derived polyclonal antibodies that bind to a human tumour necrosis factor α (TNFα).

4. The antibody composition for use according to claim 1, use according to claim 2 or method according to claim 3, wherein the blood-derived polyclonal antibodies are ovine blood-derived polyclonal antibodies.

5. The antibody composition for use according to claim 1, use according to claim 2 or method according to claim 3, wherein the blood-derived polyclonal antibodies are equine blood-derived polyclonal antibodies.

6. The antibody composition for use, use or method according to any one of the preceding claims, wherein at least 5% of the total antibodies present in the composition bind to TNFα.

7. The antibody composition for use, use or method according to any one of the preceding claims, wherein at least 10% of the total antibodies present in the composition bind to TNFα.

8. The antibody composition for use, use or method according to any one of the preceding claims, wherein the antibody composition or medicament is formulated as a solid, preferably as a suppository.

9. The antibody composition for use, use or method according to claim 8, wherein the antibodies are present at a concentration of 0.2-20% w/w, preferably wherein the antibodies are present at a concentration of 0.2-10% w/w (more preferably about 1-2% w/w or 2-6% w/w).

10. The antibody composition for use, use or method according to any one of the preceding claims, wherein a single dose comprises 1000 mg or less of the antibodies (e.g. 500 mg or less).

11. The antibody composition for use, use or method according to any one of the preceding claims, wherein a single dose comprises 250 mg or less of the antibodies.

12. The antibody composition for use, use or method according to any one of the preceding claims, wherein the antibody composition is administered to a subject at least once daily.

13. The antibody composition for use, use or method according to any one of the preceding claims, wherein the antibody composition is administered to a subject at least twice daily, preferably three times daily.

14. The antibody composition for use, use or method according to any one of the preceding claims, wherein the antibody composition is administered rectally.

15. The antibody composition for use, use or method according to any one of the preceding claims, wherein the proctitis is idiopathic proctitis.

16. The antibody composition for use, use or method according to any one of the preceding claims, wherein the subject also has a sexually transmitted disease, coeliac disease or a combination thereof.

17. The antibody composition for use, use or method according to any one of the preceding claims, wherein a subject treated for proctitis is undergoing chemotherapy, radiotherapy or a combination thereof.

18. A solid composition for treating proctitis comprising:
a. intact blood-derived polyclonal antibodies that bind to a human tumour necrosis factor α (TNFα); and
b. one or more of: gelatin, cocoa butter, polyethylene glycol, and a suppository wax.

19. The solid composition according to claim 18, wherein said solid composition is a suppository.

20. The solid composition according to claim 18 or 19, wherein the antibodies are present at a concentration of 0.2-20% w/w, preferably wherein the antibodies are present at a concentration of 0.2-10% w/w (more preferably about 1-2% w/w or 2-6% w/w).

21. The solid composition according to any one of claims 18-20, wherein the solid composition comprises:
a. intact blood-derived polyclonal antibodies that bind to a human tumour necrosis factor α (TNFα); and
b. gelatin.

22. A liquid composition (preferably an enema composition) for treating proctitis comprising:
a. intact blood-derived polyclonal antibodies that bind to a human tumour necrosis factor α (TNFα); and
b. one or more (preferably all) of: an antimicrobial, a suspension stability agent, an antifoaming agent, and a suspending agent;
wherein the composition does not comprise means for protecting the antibodies during gastrointestinal transit.

23. The solid or liquid composition according to any one of claims 18-22, wherein the blood-derived polyclonal antibodies are ovine blood-derived polyclonal antibodies.

24. The solid or liquid composition according to any one of claims 18-22, wherein the blood-derived polyclonal antibodies are equine blood-derived polyclonal antibodies.

25. The solid or liquid composition according to any one of claims 18-24, wherein at least 5% of the total antibodies present in the composition bind to TNFα, preferably wherein at least 10% of the total antibodies present in the composition bind to TNFα.

26. The antibody composition for use, use, method, solid or liquid composition according to any one of the preceding claims, wherein the antibodies or composition have not been affinity purified.

27. The antibody composition for use, use, method, solid or liquid composition according to any one of the preceding claims, wherein the antibodies or composition have been subjected to a precipitation (and optionally filtration) step only.

28. The antibody composition for use, use, method, solid or liquid composition according to any one of the preceding claims, wherein the composition is a blood serum composition that has been subjected to a precipitation (and optionally filtration) step only.

29. The antibody composition for use, use, method, solid or liquid composition according to one of the preceding claims, wherein the composition is an ovine blood serum composition comprising intact blood-derived ovine polyclonal antibodies that bind to a human TNFα and that has been subjected to a precipitation (and optionally filtration) step only.

30. The antibody composition for use, use, method, solid or liquid composition according to any one of the preceding claims, wherein the composition is a blood plasma composition that has been subjected to a precipitation (and optionally filtration) step only.

31. The antibody composition for use, use, method, solid or liquid composition according to any one of the preceding claims, wherein the composition is an equine blood plasma composition comprising intact blood-derived equine polyclonal antibodies that bind to a human TNFα and that has been subjected to a precipitation (and optionally filtration) step only.

32. The antibody composition for use, use, method, solid or liquid composition according to any one of claims 27-31, wherein the precipitation is a caprylic acid precipitation.

33. The antibody composition for use, use, method, solid or liquid composition according to any one of claims 27-32, wherein the filtration comprises the use of a 0.2 um filter and optionally a 0.45 um filter (preferably consists of the use of a glass microfiber (0.45 um) and a 0.2 um filter).


SEQUENCE LISTING (13 sequences)

SEQ ID NO: 1 - protein, 157 aa, Homo sapiens [sequence omitted]

SEQ ID NO: 2 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 3 - protein, 146 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCP

SEQ ID NO: 4 - protein, 70 aa, Homo sapiens
VRSSSRTPSDKPVAHVVANPQAEGQLQWLNRRANALLANGVELRDNQLVVPSEGLYLIYSQVLFKGQGCP

SEQ ID NO: 5 - protein, 8 aa, Homo sapiens
VRSSSRTP

SEQ ID NO: 6 - protein, 18 aa, Homo sapiens
HVVANPQAEGQLQWLNRR

SEQ ID NO: 7 - protein, 6 aa, Homo sapiens
NGVELR

SEQ ID NO: 8 - protein, 5 aa, Homo sapiens
VPSEG

SEQ ID NO: 9 - protein, 7 aa, Homo sapiens
CPSTHVL

SEQ ID NO: 10 - protein, 11 aa, Homo sapiens
ISRIAVSYQTK

SEQ ID NO: 11 - protein, 13 aa, Homo sapiens
PCQRETPEGAEAK

SEQ ID NO: 12 - protein, 16 aa, Homo sapiens
DRLSAEINRPDYLDFA

SEQ ID NO: 13 - protein, 233 aa, Homo sapiens [sequence omitted]